(19) United States
(12) Patent Application Publication
(10) Pub. No.: US 2010/0076544 A1
(43) Pub. Date: Mar. 25, 2010

(54) BIODEGRADABLE VASCULAR SUPPORT

(76) Inventors: Erika Hoffmann, Eschweiler (DE); Michael Hoffmann, Eschweiler (DE); Roland Horres, Stolberg (DE)

Correspondence Address: MEYERTONS, HOOD, KIVLIN, KOWERT & GOETZEL, P.C., P.O. BOX 398, AUSTIN, TX, 78767-0398, US

(21) Appl. No.: 12/520,976

(22) PCT Filed: Jan. 30, 2008
(86) PCT No.: PCT/DE2008/000160

§ 371 Date: Nov. 16, 2009

(30) Foreign Application Priority Data
Jan. 30, 2007 (DE) 10 2007 005 474.4
Jul. 24, 2007 (DE) 10 2007 034 350.9

(51) Int. Cl. A61F 2/82

(52) U.S. Cl. 623/1.15; 623/1.46

(57) ABSTRACT

The embodiments described herein are directed to biodegradable stents comprising an inner biodegradable metal scaffold and an outer polymeric coating. The biodegradable coating consists preferentially of biodegradable polymers and may additionally include at least one pharmacologically active substance such as an anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent.

### BACKGROUND OF INVENTION

1. Field of the Invention

The present invention is directed to biodegradable stents comprising an inner biodegradable metal scaffold and an outer polymeric coating. The biodegradable coating consists preferably of biodegradable polymers and further may contain at least one pharmacologically active substance such as an anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent.

2. Description of the Relevant Art

Nowadays, the implantation of stents is a common surgical procedure for the treatment of stenoses. Recent investigations have shown that vascular stenoses don't have to be dilated permanently by means of an endoprosthesis, particularly a stent. It is sufficient to dilate the tissue temporarily by means of an endoprosthesis since in the presence of a stent prosthesis the tissue can regenerate in the section of the vascular stenosis and then remain dilated even without the support of for example a stent. This means that after a certain time of the prosthesis supporting the tissue the prosthesis loses its effect substantially since the regenerated tissue is reenabled to maintain the normal vessel diameter by its own such that no restenosis would occur after removing the prosthesis.

A bioresorbable metal stent largely made of magnesium is disclosed in the European patent EP 1 419 793 B1. The German patent application DE 102 07 161 A1 describes stents made of magnesium alloys and zinc alloys. Bioresorbable stents made of magnesium, calcium, titanium, zirconium, niobium, tantalum, zinc or silicon or of alloys or mixtures of the aforesaid substances are disclosed in the German patent application DE 198 56 983 A1. Examples are given expressively for stents made of a zinc/calcium alloy.

Further bioresorbable metal stents made of magnesium, titanium, zirconium, niobium, tantalum, zinc and/or silicon as component A and lithium, sodium, potassium, calcium, manganese and/or iron as component B are described in the European patent application EP 0 966 979 A2. Examples are given expressively for stents made of a zinc/titanium alloy with a percentage of weight of titanium from 0.1 to 1% and of a zinc/calcium alloy with a weigh percentage of zinc to calcium of 21:1.

On the one hand these stents have the disadvantage of dissolving too rapidly and furthermore in an uncontrolled manner so that some of them already disintegrated after two weeks.

Another disadvantage of these stents is the needed degree of rigidity of these segments which is predetermined by the material, with the consequence that the stent struts have a broader and also thicker design in comparison with common stent materials as medical stainless steel, Nitinol and cobalt/chromium stents. The result is a larger contacting surface with the surrounding, on the other hand the stent extends further into the lumen and may influence blood flow. Also the incorporation into the vascular wall is delayed thereby because of the larger surface to be covered.

Since furthermore the dissolution process begins before the incorporation of the stent into the vascular wall fragments may detach which are going to be transported through the bloodstream and thus may cause a cardiac infarction.

A further disadvantage of the described bioresorbable metal stents is that they only provide very limited facilities of integrating a pharmacologically active agent into the metal scaffold which shall be released during the degradation of the stent.

### SUMMARY OF THE INVENTION

The embodiments disclosed herein provide a stent which exerts its support function only for the time until the regenerated tissue is reenabled to assume this function and avoids the disadvantages of conventional stents.

This objective is solved by the technical teaching of the independent claims. More advantageous embodiments result from the dependent claims, the description and the examples.

In one embodiment, a biodegradable stent comprises an inner bioresorbable scaffold containing at least one metal and a polymeric biodegradable coating substantially surrounding the inner bioresorbable scaffold. The inner bioresorbable scaffold containing at least one metal biodegrades at a rate that is faster than the biodegredation rate of the polymeric outer wrapper.

In one embodiment, a biodegradable stent consists of an inner bioresorbable scaffold containing at least one metal and a polymeric biodegradable coating substantially surrounding the inner bioresorbable scaffold. The inner bioresorbable scaffold containing at least one metal biodegrades at a rate that is faster than the biodegredation rate of the polymeric outer wrapper.

### DETAILED DESCRIPTION OF THE PREFERRED EMBODIMENTS

Described herein are biodegradable stents comprising an inner bioresorbable scaffold containing at least one metal, surrounded by a polymeric biodegradable coating.

The polymeric layer is reduced by itself on the stent struts or may wrap the complete cavity like a stocking, either on the abluminal or on the luminal side of the stent body, or may fill the free interspaces of the stent body in such a way that the wrapper lies on the same plane as the likewise wrapped stent struts. The forms of coating can be usefully combined.

According to an embodiment the inner scaffold of the stent consists of a metal, a metal alloy, metal oxide, metal salt, metal carbide, metal nitride or a mixture of said substances.

Particularly preferred is that the inner scaffold consists of a metal alloy containing up to 30% percentage of weight, preferably up to 20% percentage of weight and particularly preferably only up to 10% percentage of weight of metal oxides, metal salts, metal carbides and/or metal nitrides. Furthermore, up to 1% percentage of weight of other components such as carbon, nitrogen, oxygen, contaminations, non-metals or organic substances can be included in the composition or the alloy.

Furthermore, the inner metal scaffold has the property of dissolving more rapidly than the polymeric outer coating, i.e. the inner structure of the stent undergoes more rapidly biodegradation than the polymeric coating under physiological conditions. When using different biodegradable polymers on the stent there is the further option to use polymers which differ in degradation time. Thus it can be advantageous that the luminal coating dissolves slower than the abluminal stent coating. For example the stent degradation by the blood stream is thus delayed. Another advantage is the stabilization of the stent body so that no fragments can detach prematurely. A complete wrapping all over the inner surface of the stent body may further regulate these effects.

Preferably the metal alloy is converted inside the polymeric wrapper into their corresponding metal salts which can pass out through the polymeric coating.

Suitable metallic inner scaffolds of the stent are made of metallic materials displaying a potential difference of at least −0.48 eV, preferred at least −0.53 eV, more preferred at least −0.58 eV and particularly preferred at least −0.63 eV in comparison to the calomel electrode, or displaying a potential difference in the range of −0.3 to −2.5 eV, preferred from −0.4 to −1.5 eV, more preferred from −0.45 to −1.25 eV and particularly from −0.5 to −1.0 eV in comparison to the calomel electrode.

In order to register the measured potential differences an electrochemical disposal of two half cells is used. As the potential difference shall be determined in a reproducible manner a point of reference is needed which shall change during the measurement.

To this aim second kind electrodes are used in general. These metal electrodes are covered with their insoluble salts and a salt solution of a higher concentration flows around them. To this group belong for example the calomel electrode (correctly: saturated calomel electrode, SCE). The name “calomel” is derived from the trivial name of the not readily soluble mercury(I) chloride.

The calomel electrode (as well as some other metal/metal salt electrodes) have proved themselves in practice as reference electrodes. For example, a practical application is the measurement of a potential difference in a solution by means of a calomel electrode. Such a measurement can also be used for determining a suitable metal, respectively a suitable metal alloy.

The potential difference is commonly described by the known Nernst equation:

$${E}_{\frac{{Hg}_{2}^{2 +}}{Hg}} = {E}_{{{Hg}_{2}^{2 +}}_{Hg}}^{0} + \frac{0 , 059 V}{2} \cdot 1 g {c}_{{Hg}_{2}^{2 +}}$$

As can be easily seen, potential E depends exclusively on the concentration of the not readily soluble mercury salt. If the anion concentration, i.e. the counterion concentration, is held constant also E remains constant. This can be achieved by choosing a very high anion concentration.

The calomel electrode consists of mercury, the electrode itself, covered with solid Hg₂Cl₂ and dipping into a saturated KCl solution (high concentration of Cl⁻ ions). The salt bridge is used for exact measurements in order to suppress diffusion potentials. Tables containing values determined by such a setup must always be tabulated against this reference point (calomel electrode).

Thus the calomel electrode as a second kind electrode is highly suitable as a reference electrode for potential measurements. The calomel electrode is also chosen as reference electrode.

The setup drafted above can now be used to choose suitable materials which are less noble than calomel, i.e. their reference potential in the range of 0.3 to 2.5 eV, preferred 0.35 to 2.2 eV, more preferred 0.4 to 1.8 eV, more preferred 0.45 to 1.4 eV, more preferred 0.48 to 1.2 eV, more preferred 0.50 to 1.0 eV, more preferred 0.50 to 0.9 eV, more preferred 0.50 to 0.80 eV and particularly preferred 0.50 to 0.70 eV (given as absolute values, i.e. without an algebraic sign) in comparison to the calomel electrode.

Particularly preferred is that the inner scaffold consists of an alloy containing magnesium, calcium, manganese, iron, zinc, silicon, yttrium, zirconium and/or gadolinium, and more preferred that magnesium, calcium, manganese, iron, zinc, silicon, yttrium, zirconium or gadolinium account for the higher percentage of weight—indicated as % by weight—in this alloy.

In order to avoid that the metal scaffold dissolves too rapidly and disintegrates into fragments which can be washed away from the bloodstream and cause a heart infarction the inner bioresorbable scaffold of metal, metal salt, metal oxide and/or metal alloy is enclosed in a polymeric coating covering the stent struts or, as already mentioned, the complete cylindrical stent body.

According to an embodiment the polymeric coating is realized in such a way that the inner metal scaffold can dissolve itself inside the coating and the metal ions can pass out of the coating into the surrounding tissue. Thus the polymeric coating is porous, or provided with channels or openings and realized in such a way that ions (anions as well as cations) can pass out.

According to an embodiment the polymeric coating can be provided in form of an ion-permeable membrane or can have nano- to micro-pores which enable the permeation of water as well as the passing out of ions.

Such coatings, porous or provided with channels or openings, can be obtained by applying either a polymeric coating onto the stent which leads to a permeable polymeric layer, or by rendering the polymeric coating permeable after the application. The term “permeable” shall mean that a polymeric coating is porous or has channels, pores or openings which enable the entry of water and the escape of ions.

Such coatings can be obtained either through polymers which lead to a porous coating on the stent surface by themselves, or through a solution of oligomers and/or polymers which is applied onto the stent surface and wherein the oligomers and/or polymers undergo a further cross-linking (for example by glutaraldehyde or other dialdehydes) after the application and the not cross-linked oligomers and polymers are then washed away from the coating by a solvent preferably or by the use of an autopolymerizable substance such as unsaturated fatty acids and derivatives of unsaturated fatty acids, wherein the not polymerized substances are preferably washed away from the stent surface by a solvent. Further options for generating a permeable polymeric coating on the stent are the application of a comparatively unflexible or rigid, respectively brittle, polymeric coating which bursts on dilating the stent and forms cracks and thus becomes permeable preferably after inflating the stent. Furthermore, one or more substances can be added to the polymeric coating solution which can be washed away after applying the coating onto the stent and leave a permeable structure. Preferred herein is the addition of salts in form of powder, particles or also in solved form. The polymeric coating having formed the salts can be washed off the polymeric coating preferably by water and leave a porous structure. Of course the salts don't have to be washed off before implanting the stent. The stent with a polymeric coating together with all included pharmacologically acceptable salts can also be implanted in a not yet permeable form and the salts are then naturally washed off through the bloodstream whereas a permeable coating is obtained only after the implant when the physiologically acceptable salts such as NaCl, NaBr, NaI, NaSO₄, KCl, NaHCO₃ or other physiologically acceptable salts known to the person skilled in the art are washed off the polymeric coating.

Finally, there's also the option of generating a non-permeable polymeric coating on the stent which then is rendered permeable through chemical, mechanic, optic or other methods. For example, the use of bases or acids can render the polymeric coating permeable, as well as the use of lasers or of other mechanical polishing methods such as chemical polishing or sandblast methods. Such methods are known to the person skilled in the art and of course have to be adjusted to the respective coating, its thickness and hardness and to the used polymers.

By this embodiment it is ensured that at least in the beginning a metal-containing inner scaffold is provided which can exert sufficient spreading force to the vessel to keep it open and to avoid a spontaneous recoil, i.e. a spontaneous collapsing of the vessel after dilation because of damaged or relaxed vascular muscles. Since a vessel can regain its elasticity and resilience after a certain time a stent as a permanent implant, i.e. as a non- or only slowly biodegradable implant, isn't necessary to keep the vessel permanently open.

Moreover, there is the problem of restenosis or in-stent stenosis in non-biodegradable stents, whereas the vessel is constricted or occluded inside the stent through overgrowing of the stent with smooth muscle cells. Further there is the problem to place another stent at a section where a non-biodegradable stent was already implanted.

Furthermore there is the danger of late thrombosis when using substance-releasing stents made from the known non-biodegradable materials which can lead spontaneously often even after one year to an acute occlusion. These worrying results were made public in the summer of 2006. The stent surface which is still not integrated because of the cytostatic actions of the active agent was identified as the cause of the late thromboses massively occurring after this time. The benefit occurring after the use of substance-releasing stents was and is still severely questioned thereby.

Also these disadvantages are avoided by the stent as it dissolves completely in a controlled manner after a certain time. The polymeric wrapper enables the biologic degradation of the metal inner scaffold without the danger of fragments being detached since the polymeric wrapper covers the inner scaffold entirely in such a way that larger or also smaller fragments cannot permeate through the polymeric coating. In contrast the permeation of ions and salts is possible which are formed from the metal scaffold under physiological conditions.

Such metal ions as well as their counterions can permeate through the polymeric coating, respectively escape through the nano- to micro-pores.

In a particularly preferred embodiment the inner metal or metal-containing scaffold is degraded more rapidly under physiological conditions than the outer polymeric wrapper so that the void polymeric wrapper grown into the vascular wall remains there for a certain time but however is flexible, does not exert anymore a significant pressure onto the vascular wall and even fits closely to the new vessel shape. Then also this polymeric wrapper is biodegraded so that after 2 to 12 months the biodegradable stent is completely dissolved. Thus the polymeric coating dissolves slower than the metal inner structure and enables the permeation of salts and ions so that the inner structure can dissolve and the salts and ions can be resorbed from the surrounding tissue. In this particularly preferred embodiment the coated stent is designed in such a way that the stent has grown into the tissue before the bioresorbable coating starts dissolving. The dissolution of the inner stent scaffold can occur already before the stent has grown into the vascular tissue whereas it is preferred that the ingrowing and the dissolution of the inner stent scaffold substantially occur concomitantly. On the contrary, the dissolution velocity of the inner stent structure in comparison to the coating applied thereon is essential in thus particularly preferred embodiment. Preferentially the polymeric coating should be dissolved up to maximally 15% by weight, more preferably up to 10% by weight and particularly preferably up to 5% by weight when the inner stent body has dissolved completely. The term “polymeric coating” refers only to components forming the polymeric coating and not to components of the coating which are not bound in a polymeric form such as salt particles which shall be washed off the coating through the bloodstream. In other words, the dissolution velocity of the inner stent scaffold in comparison to the polymeric coating shall amount to at least 10:1, preferably to 20:1, more preferably to 30:1, further more preferably to 40:1 and particularly preferably to 50:1. The relation 20:1 herein means that at least 20% by weight of the inner stent scaffold have dissolved and have been released through the polymeric coating when maximally 1% by weight of the polymeric coating is dissolved or has been biodegraded.

A way of determining the dissolution kinetics of an uncoated metal stent consists in placing the stent on a tube between two porous membranes or filter plates and flowing physiological saline solution, PBS buffer (phosphate buffer with 14.24 g NaH₂PO₄, 2.72 g K₂HPO₄ and 9 g NaCl; pH 7.4; T=37° C.) or blood serum through the tube, preferably with a similar velocity as the bloodstream in the vessels of the human body.

The dissolution velocity of the polymeric coating can be determined by applying the polymeric coating onto a non-bioresorbable stent, e.g. a stainless steel stent, and placing it likewise between two diaphragms in a tube through which physiological saline solution, PBS buffer or blood serum is conducted.

The dissolution of the stent can be observed optically and additionally be quantified by weight measurement.

In another embodiment the polymeric coating displays holes, openings and/or channels which enable the permeation of salts or ions but are not that large that fragments of the metal inner scaffold can pass through.

These holes, openings and/or channels are preferably oriented perpendicular to the central axis of the individual stent struts and moreover, they are preferentially not disposed at the ends of the stent struts. These holes, openings and/or channels can be applied mechanically, chemically, thermically or optically to the polymer, for example by mechanical treatment such as sandblast, by chemical methods such as etching or oxidation, by mechanic-chemical methods as polishing methods, by thermal methods such as melting or branding, or by optical methods such as laser treatment.

In another particularly preferred embodiment the holes, openings and/or channels are filled with a pharmacologically active agent. Suitable active agents are listed further below. The active agent(s) to be applied into the holes, openings and/or channels can be mixed with a pharmaceutically acceptable carrier such as a salt, a contrast medium, a bulking agent, an oligomer, organic compounds such as amino acids, vitamins, carbohydrates, fatty acids, oils, fats, waxes, proteins, peptides, nucleotides or a solvent.

For example, lactose, starch, sodium carboxymethyl starch, sorbitol, sucrose, magnesium stearate, dicalcium phosphate, calcium sulfate, talc, mannitol, ethyl alcohol, polyvinyl alcohols, polyvinyl pyrrolidones, gelatine, naturally occurring sugars, naturally occurring as well as synthetic gums such as acacia gum or guar gum, sodium alginate, sodium benzoate, sodium acetate, glycerides, myristates such as isopropyl myristate, palmitates, tributyl and triethyl citrates and their acetyl derivatives, phthalates such as dimethyl phthalate or dibutyl phthalate, benzoic acid benzyl ester, triacetine, 2-pyrrolidone, boric acid, magnesium aluminium silicates, naturally occurring carob gum, gum karaya, guar, tragacanth, agar, carrageenans, cellulose, cellulose derivatives such as methyl cellulose, sodium carboxymethyl cellulose, hydroxypropyl methyl cellulose, microcrystalline cellulose as well as alginates, aluminas and bentonites, polyethylene glycol and also waxes such as beeswax, carnauba wax, candelilla wax and the like can be used as a pharmacologically acceptable carrier.

Further carriers can be vitamins such as vitamin A, vitamin C (ascorbic acid), vitamin D, vitamin H, vitamin K, vitamin E, vitamin B1, vitamin B2, vitamin B3, vitamin B5, vitamin B6, vitamin B12, thiamine, riboflavine, niacine, pyridoxine and folic acid.

Further suitable carriers are heparin, heparan sulfate, chitosan, chitin, chondroitin sulfate, collagen, fibrin, xanthones, flavonoids, terpenoids, cellulose, rayon, peptides with 50 to 500 amino acids, nucleotides with 20 to 300 base pairs as well as saccharides with 20 to 400 sugar monomers, fatty acids, fatty acid esters, fatty acid derivatives, ethers, lipids, lipoids, glycerides, triglycerides, glycol ester, glycerine ester, and oils such as linseed oil, hempseed oil, corn oil, walnut oil, rape oil, soy bean oil, sun flower oil, poppy-seed oil, safflower oil, wheat germ oil, safflor oil, grape-seed oil, evening primrose oil, borage oil, black cumin oil, algae oil, fish oil, cod-liver oil and/or mixtures of the aforementioned oils.

Suitable amino acids are glycine, alanine, valine, leucine, isoleucine, serine, threonine, phenylalanine, tyrosine, tryptophan, lysine, arginine, histidine, aspartate, glutamate, asparagine, glutamine, cysteine, methionine, proline, 4-hydroxyproline, N,N,N-trimethyllysine, 3-methylhistidine, 5-hydroxylysine, O-phosphoserine, γ-carboxyglutamate, ε-N-acetyllysine, ω-N-methylarginine, citrulline, ornithine.

Furthermore, the following fatty acids and esters of the following fatty acids are suitable carriers: Eicosapentaenoic acid (EPA), timnodonic acid, docosahexaenoic acid (DHA), α-linolenic acid, γ-linolenic acid, myristoleic acid, palmitoleic acid, petroselinic acid, oleic acid, vaccenic acid, gadoleinic acid, gondoinic acid, erucinic acid, nervonic acid, elaidinic acid, t-vaccenic acid, linoleic acid, γ-linolenic acid, dihomo-γ-linolenic acid, arachidonic acid, α-linolenic acid, stearidonic acid, DPA, meadic acid, stellaheptaenic acid, taxolic acid, pinolenic acid, sciadonic acid, taririnic acid, santalbinic or ximeninic acid, stearolinic acid, 6,9-octadeceninic acid, pyrulinic acid, crepenynic acid, heisterinic acid, ETYA, lauric acid, myristic acid, palmitic acid, margaric acid, stearic acid, arachinic acid, behenic acid and lignoceric acid as well as derivatives and mixtures of aforesaid fatty acids.

Particularly preferred, however, is to solve at least one anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent in a solvent and to apply it as a substantially pure active agent into the holes, openings and/or channels in the polymeric coating, what can be achieved via a squirting or a pipetting method. After evaporation of the solvent the active agent remains inside the holes, openings and/or channels.

Common organic solvents such as dimethyl sulfoxide, ether such as dioxane, tetrahydrofuran (THF), petroleum ether, diethylether, methyl tert-butyl ether, ketones such as acetone, butanone or pentanone, alcohols such as methanol, ethanol, propanol, iso-propanol, carbonic acids such as formic acid, acetic acid, propionic acid, amides such as dimethylformamide (DFA) or dimethylacetamide, aromatic solvents such as toluene, benzene, xylene, pure hydrocarbon solvents such as pentane, hexane, cyclohexane, halogenized solvents such as chloroform, methylene chloride, carbon tetrachloride as well as carbonic acid esters such as acetic acid methyl and acetic acid ethyl ester as well as water serve as solvent, depending on the solubility of the active agent.

Furthermore it is particularly preferred to add the active agent to a contrast medium or contrast medium analogue and apply it in this form into the holes, openings and/or channels.

As contrast media or contrast medium analogies common radiographic contrast media (positive as well as negative contrast media) can be used, such as those commonly utilized for imaging methods (arthrography, radiography, computer tomography (CT), nuclear spin tomography, magnetic resonance tomography (MRT).

Contrast media and/or contrast media analogues usually contain barium, iodine, manganese, iron, lanthanum, cerium, praseodymium, neodymium, samarium, europium, gadolinium, terbium, dysprosium, holmium, erbium, thulium, ytterbium and/or lutetium preferably as ions in the bound and/or complex form, wherein iodine containing contrast media are preferred.

The following examples can be named as iodine-containing contrast media:

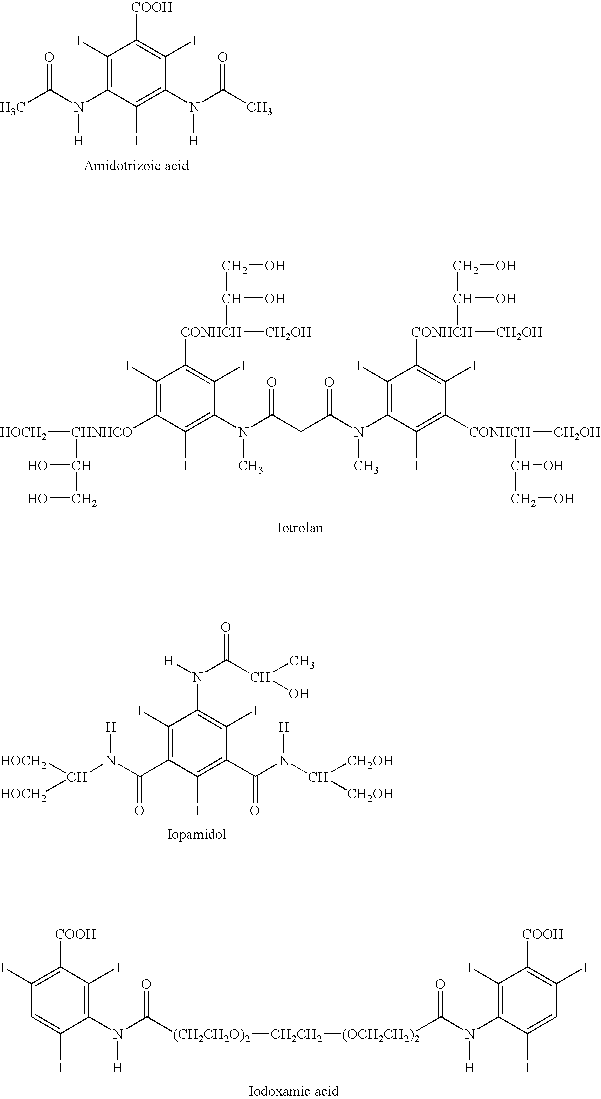

A further example is Iodine Lipiodol®, an iodized Oleum papaveris, a poppy oil. The parent substance of iodized contrast media, amidotrizoate in form of sodium and meglumine salts, is commercially available under the tradenames Gastrografin® and Gastrolux® (Germany, Switzerland).

Also gadolinium-containing or superparamagnetic iron oxide particles as well as ferrimagnetic or ferromagnetic iron particles such as nanoparticles are preferred.

Another class of preferred contrast media are paramagnetic contrast media which usually contain a lanthanoid.

Among the paramagnetic substances with unpaired electrons are for example gadolinium (Gd³⁺), having seven unpaired electrons in total. Furthermore belong to this group europium (Eu²⁺, Eu³⁺), dysprosium (Dy³⁺) and holmium (Ho³⁺). These lanthanoids can also be used in a chelated form by utilizing for example hemoglobin, chlorophyll, polyaza acids, polycarbonic acids and particularly EDTA, DTPA, DMSA, DMPS and DOTA as chelators.

Examples for gadolinium-containing contrast media are gadolinium and diethylenetriamine pentaacetic acid.

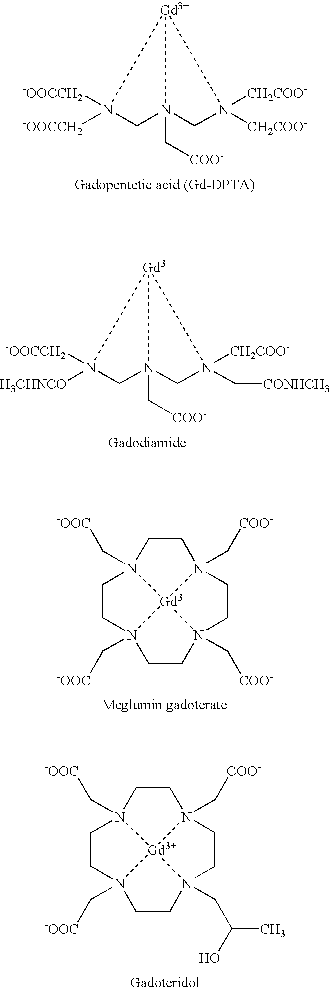

For augmenting the transfer of active agent so-called transport mediators can be used preferentially which, however, can also be the active agent itself. Of special interest as transport mediators are low molecular chemical compounds that accelerate or facilitate the uptake of active agents into the vascular wall so that the present active agent or combination of active agents can be transferred in a controlled manner and in the provided dosage during the short-term contact.

Such properties are found in substances interacting directly with the lipid double layer of the cell membrane or with receptors on the cell membrane, or entering the cytosol via membrane transport proteins acting as carriers or channels (ion pumps) where they change the membrane potential and thus the membrane permeability of the cells. The uptake of an active agent into the cells is thus facilitated, respectively accelerated.

To such useful compounds belong for example vasodilators such as bradykinin, kallidin, histamine or NOS-synthase which releases vasodilatory NO from L-arginin, substances of herbal origin such as the extract of gingko biloba, DMSO, xanthones, flavonoids, terpenoids, herbal and animal dyes, food colorants, NO-releasing substances such as pentaerythrytiltetranitrate (PETN). The aforementioned contrast media and contrast medium analogues belong also to this category.

The holes, openings and/or channels are filled with an active agent or a composition of active agents in such a way that the content is dissolved relatively rapidly and released, thus uncovering or opening the holes, openings and/or channels directly after the stent implant. The active agent inside the holes, openings and/or channels is released very rapidly so that it can be characterized as a fast release, i.e. a rapid release which takes preferably a few hours up to 2 days.

The problem of restenosis, respectively the directed ingrowing of the stent into the vascular wall can thus be controlled via an initial release of an active agent.

This rapid release of active agent can be further combined with a slow release of active agent whereas this can be the same or another active agent. This active agent is applied into the polymeric coating so that the polymeric coating also acts as a carrier.

Preferably, a cytostatic dosage of an anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent is contained in the polymeric coating. This active agent is then released in a measure corresponding to the biodegradation of the polymeric coating.

Thus the bioresorbable stent additionally allows for the option of a release of an active agent, and especially for the combination of a rapid and a slow release of active agent. Additionally, active agents counteracting platelet adhesion respectively thrombus formation can be used in a directed manner by wrapping the stent body on the luminal side. Such options allow for a directed release of an active agent or a combination of active agents which is specifically adapted to the surrounding. The active agents can be used on the same stent in a directed manner and independently from one another.

Thus the stent offers a number of decisive advantages in respect of known embodiments. First, the polymeric wrapper prevents the disintegration and bursting of the metal scaffold which may lead to serious consequences. In the particularly preferred embodiments the faster biodegradation of the inner metal or metal-containing scaffold in comparison to the polymeric coating ensures that the inner scaffold dissolves first and its dissolution products are released in a controlled manner and resorbed by the tissue. When the vessel can resume its proper support the inner scaffold is already in the state of dissolution. After dissolution of the inner structure also the polymeric outer wrapper is biodegraded.

Because of the structure of the polymeric outer wrapper with holes, openings, channels and/or pores a system is obtained additionally which combines a rapid and a slow release of active agent or of a combination of active agents in a directed manner.

The holes, openings, channels and/or pores can be filled with an active agent or a composition containing an active agent in a directed manner and the active agent can be released rapidly from these cavities, or the entire surface or a part of the surface of the outer polymeric wrapper is coated with an active agent or a composition containing an active agent. Herein, any embodiment can be conceived and realized.

Furthermore, there is the option of embedding one or also more active agents into the polymeric biodegradable layer which then will be slowly released in the same degree as the polymeric outer wrapper is dissolved, i.e. biodegraded.

The system is very flexible, offers the advantages of a conventional drug-eluting stent and additionally combines a fast treatment with an active agent with a local long-term therapy and furthermore is completely biodegradable so that after a certain time no foreign body is present anymore in the patient's body. For example, the problem of late stent restenosis which currently worries experts can thus be avoided to a 100%.

For example, the resorbable stent may consist at least 30% by weight, preferred at least 40% by weight, more preferred at least 50% by weight, more preferred at least 60% by weight, more preferred at least 70% by weight, more preferred at least 80% by weight and particularly preferred at least 90% by weight of the metal zinc, calcium, manganese or iron.

It is further preferred that the implant additionally displays 0-60% by weight, preferred 0.01-59% by weight, more preferred 0.1-59%, still more preferred 0.1-58% by weight calcium. Particularly preferred, the mass of calcium is in the range of 1.5-50% by weight, 2.0-40% by weight, 2.5-30% by weight, 3.0-20& by weight and particularly preferred 3.5-10% by weight.

Instead of calcium or a combination with calcium the implant may contain 0-80% by weight, preferred 0.01-70% by weight, more preferred 0.1-60% by weight, more preferred 1-50% by weight magnesium. Preferably, the mass of magnesium is in the range of 0.1-80% by weight, 5.0 to 70% by weight, 7.5 to 60% by weight, 10.0-50% by weight and particularly preferred in the range of 20-40% by weight.

In addition to zinc and/or iron and optionally calcium and/or magnesium, an inventive stent may further contain at least one metal selected from the group comprising lithium, sodium, magnesium, aluminum, potassium, calcium, scandium, titanium, vanadium, chromium, manganese, iron, cobalt, nickel, copper, zinc, gallium, silicon, yttrium, zirconium, niobium, molybdenum, technetium, ruthenium, rhodium, palladium, silver, indium, tin, lanthanum, cerium, praseodymium, neodymium, promethium, samarium, europium, gadolinium, terbium, dysprosium, holmium, erbium, thulium, ytterbium, lutetium, tantalum, tungsten, rhenium, platinum, gold, lead and/or at least one metal salt with a cation selected from the group comprising Li⁺, Na⁺, Mg²⁺, K⁺, Ca²⁺, Sc³⁺, Ti⁴⁺, V²⁺, V³⁺, V⁴⁺, V⁵⁺, Cr²⁺, Cr³⁺, Cr⁴⁺, Cr⁶⁺, Mn²⁺, Mn³⁺, Mn⁴⁺, Mn⁵⁺, Mn⁶⁺, Mn⁷⁺, Fe²⁺, Fe³⁺, Co²⁺, Co³⁺, Ni²⁺, Cu⁺, Cu²⁺, Zn²⁺, Ga⁺, Ga³⁺, Al³⁺, Si⁴⁺, Y³⁺, Zr²⁺, Zr⁴⁺, Nb²⁺, Nb⁴⁺, Nb⁵⁺, Mo⁴⁺, Mo⁶⁺, Tc²⁺, Tc³⁺, Tc⁴⁺, Tc⁵⁺, Tc⁶⁺, Tc⁷⁺, Ru³⁺, Ru⁴⁺, Ru⁵⁺, Ru⁶⁺, Ru⁷⁺, Ru⁸⁺, Rh³⁺, Rh⁴⁺, Pd²⁺, Pd³⁺, Ag⁺, In⁺, In³⁺, Ta⁴⁺, Ta⁵⁺, W⁴⁺, W⁶⁺, Pt²⁺, Pt³⁺, Pt⁴⁺, Pt⁵⁺, Pt⁶⁺, Au⁺, Au³⁺, Au⁵⁺, Sn²⁺, Sn⁴⁺, Pb²⁺, Pb⁴⁺, La³⁺, Ce³⁺, Ce⁴⁺, Gd³⁺, Nd³⁺, Pr³⁺, Tb³⁺, Pr³⁺, Pm³⁺, Sm³⁺, Eu²⁺, Dy³⁺, Ho³⁺, Er³⁺, Tm³⁺, Yb³⁺. In addition to the aforementioned metals and metal salts which taken together are present in the amount of less than 5% by weight small amounts of non-metals, carbon, sulfur, nitrogen, oxygen and/or hydrogen may be present

Particularly the presence of yttrium in amounts of 0.01-10% by weight, preferred 0.1-9% by weight, more preferred 0.5 to 8% by weight, more preferred 1.0 to 7.0% by weight, more preferred 2.0 to 6.0% by weight and particularly preferred 3.0 to 5.0% by weight can be advantageous.

A preferred composition of an inventive implant comprises for example

| | |
|---|---|
| 50% (w/w)-90% (w/w) | of zinc |
| 0.0% (w/w)-50% (w/w) | of magnesium |
| 0.0% (w/w)-50% (w/w) | of calcium |
| 0.0% (w/w)-10% (w/w) | of yttrium |
| 0.0% (w/w)-10% (w/w) | of rare earths |
| 0.0% (w/w)-5% (w/w) | of other metals, metal salts, non-metals, |
| | carbon, sulphur, nitrogen, oxygen, hydrogen. |

The carbon, sulphur, nitrogen, oxygen, hydrogen or other non-metals or semi-metals may be present in form of anions and/or polymers.

Further preferred compositions are:

| | |
|---|---|
| 55% (w/w)-100% (w/w) | of zinc |
| 0.1% (w/w)-40% (w/w) | of magnesium |
| 0.1% (w/w)-40% (w/w) | of calcium |
| 0.01% (w/w)-9% (w/w) | of yttrium |
| 0.01% (w/w)-7% (w/w) | of rare earths |
| 0.01% (w/w)-4% (w/w) | of other metals, metal salts, non-metals, |
| | carbon, sulphur, nitrogen, oxygen, hydrogen. |
| 75% (w/w)-95% (w/w) | of zinc |
| 0.01% (w/w)-15% (w/w) | of magnesium |
| 0.01% (w/w)-15% (w/w) | of calcium |
| 0.01% (w/w)-6% (w/w) | of yttrium |
| 0.01% (w/w)-3% (w/w) | of rare earths |
| 0.01% (w/w)-2% (w/w) | of other metals, metal salts, non-metals, |
| | carbon, sulphur, nitrogen, oxygen, hydrogen. |
| 41% (w/w)-91% (w/w) | of zinc |
| 7.0% (w/w)-55% (w/w) | of magnesium |
| 0.00% (w/w)-10% (w/w) | of calcium |
| 0.00% (w/w)-6% (w/w) | of yttrium |
| 0.01% (w/w)-2% (w/w) | of rare earths, other metals, metal salts, |
| | non-metals, carbon, sulphur, nitrogen, |
| | oxygen, hydrogen. |
| 30% (w/w)-93% (w/w) | of zinc |
| 0.00% (w/w)-10% (w/w) | of magnesium |
| 2.0% (w/w)-69% (w/w) | of calcium |
| 0.00% (w/w)-6% (w/w) | of yttrium |
| 0.01% (w/w)-2% (w/w) | of rare earths, other metals, metal salts, |
| | non-metals, carbon, sulphur, nitrogen, |
| | oxygen, hydrogen. |
| 55% (w/w)-100% (w/w) | of iron |
| 0.1% (w/w)-40% (w/w) | of magnesium |
| 0.1% (w/w)-40% (w/w) | of calcium |
| 0.01% (w/w)-9% (w/w) | of yttrium |
| 0.01% (w/w)-7% (w/w) | of rare earths |
| 0.01% (w/w)-4% (w/w) | of other metals, metal salts, non-metals, |
| | carbon, sulphur, nitrogen, oxygen, hydrogen. |
| 55% (w/w)-100% (w/w) | of iron |
| 0.1% (w/w)-40% (w/w) | of zinc |
| 0.1% (w/w)-40% (w/w) | of calcium |
| 0.01% (w/w)-9% (w/w) | of yttrium |
| 0.01% (w/w)-7% (w/w) | of rare earths |
| 0.01% (w/w)-4% (w/w) | of other metals, metal salts, non-metals, |
| | carbon, sulphur, nitrogen, oxygen, hydrogen. |
| 55% (w/w)-100% (w/w) | of iron |
| 0.1% (w/w)-40% (w/w) | of zinc |
| 0.1% (w/w)-40% (w/w) | of magnesium |
| 0.01% (w/w)-9% (w/w) | of yttrium |
| 0.01% (w/w)-7% (w/w) | of rare earths |
| 0.01% (w/w)-4% (w/w) | of other metals, metal salts, non-metals, |
| | carbon, sulphur, nitrogen, oxygen, hydrogen. |
| 0.1% (w/w)-30% (w/w) | of iron |
| 0.1% (w/w)-30% (w/w) | of zinc |
| 0.1% (w/w)-30% (w/w) | of calcium |
| 0.1% (w/w)-30% (w/w) | of magnesium |
| 0.01% (w/w)-10% (w/w) | of yttrium |
| 0.01% (w/w)-4% (w/w) | of rare earths |
| 0.01% (w/w)-4% (w/w) | of other metals, metal salts, non-metals, |
| | carbon, sulphur, nitrogen, oxygen, hydrogen. |
| 55.0% (w/w)-75.0% (w/w) | of magnesium |
| 10.0% (w/w)-20.0% (w/w) | of calcium |
| 5.0% (w/w)-15.0% (w/w) | of yttrium |
| 5.0% (w/w)-10.0% (w/w) | of other metals, metal salts or rare earths |
| 0.5% (w/w)-10.0% (w/w) | of non-metals, carbon, sulphur, nitrogen, |
| | oxygen, hydrogen. |
| 20.0% (w/w)-40.0% (w/w) | of magnesium |
| 20.0% (w/w)-40.0% (w/w) | of calcium |
| 20.0% (w/w)-40.0% (w/w) | of zinc |
| 0.0% (w/w)-5.0% (w/w) | of yttrium |
| 0.1% (w/w)-5.0% (w/w) | of other metals, metal salts or rare earths |
| 0.1% (w/w)-5.0% (w/w) | of non-metals, carbon, sulphur, nitrogen, |
| | oxygen, hydrogen. |
| 80% (w/w)-95% (w/w) | of magnesium |
| 0.0% (w/w)-4% (w/w) | of calcium |
| 0.1% (w/w)-4% (w/w) | of yttrium |
| 0.0% (w/w)-4% (w/w) | of other metals, metal salts or rare earths |
| 0.1% (w/w)-4% (w/w) | of non-metals, carbon, sulphur, nitrogen, |
| | oxygen, hydrogen. |

For the listed compositions it is evident that the sum of all components must add up to 100.00% by weight.

The term “other metals” refers preferably to titanium, zirconium, niobium, tantalum, silicon, lithium, sodium, potassium and manganese, and “non-metals” preferably to carbon, nitrogen and oxygen.

The term “resorbable” as used herein means that the implant is slowly dissolved in the organism for a certain time and at some point only its degradation products are present in the body in a dissolved form. At this point solid components or fragments of the implant don't exist anymore. The degradation products should be substantially harmless in physiological terms and lead to ions or molecules which either occur in the organism anyway, or can be degraded by the organism to harmless substances, or can be excreted.

Metals that can be used in combination with zinc are preferably the following: lithium, sodium, magnesium, aluminum, potassium, calcium, scandium, titanium, vanadium, chromium, manganese, iron, cobalt, nickel, copper, zinc, gallium, silicon, yttrium, zirconium, niobium, molybdenum, technetium, ruthenium, rhodium, palladium, silver, indium, tin, lanthanum, cerium, praseodymium, neodymium, promethium, samarium, europium, gadolinium, terbium, dysprosium, holmium, erbium, thulium, ytterbium, lutetium, tantalum, tungsten, rhenium, platinum, gold, lead. Particularly preferred are magnesium, calcium, iron, yttrium. Further preferred are combinations of zinc with or without one of the aforementioned metals together with metal salts. Such combinations can be described as metal salt-containing molten zinc baths or as metal salt-containing zinc alloys. The content of metal salts may only be that large that a sufficient flexibility of the material is ensured. The expandability shouldn't be significantly compromised neither. Suitable metal salts are those mentioned further below and particularly salts of magnesium, calcium, iron and yttrium.

Better than the use of metals is, however, the use of resorbable alloys which for example may contain for example the following metals together with zinc: lithium, sodium, magnesium, aluminum, potassium, calcium, scandium, titanium, vanadium, chromium, manganese, iron, cobalt, nickel, copper, zinc, gallium, silicon, yttrium, zirconium, niobium, molybdenum, technetium, ruthenium, rhodium, palladium, silver, indium, tin, lanthanum, cerium, praseodymium, neodymium, promethium, samarium, europium, gadolinium, terbium, dysprosium, holmium, erbium, thulium, ytterbium, lutetium, tantalum, tungsten, rhenium, platinum, gold, lead. Such metals are partially included only in small amounts.

Preferred are magnesium/zinc alloys containing zinc in the range of 10 to 78% by weight, preferred 25 to 68% by weight and particularly preferred 36 to 53% per weight. It is further preferred that this magnesium/zinc alloy additionally contains scandium, titanium, vanadium, yttrium, zirconium, niobium, molybdenum, technetium, ruthenium, rhodium, palladium, silver or indium, and particularly yttrium in an amount of 0.3-11, preferred 0.7-10, more preferred 1.1-8.5 and particularly preferred 2-7% per weight.

Further preferred are alloys containing apart of zinc mainly calcium, magnesium, iron, tin, zinc or lithium, together with up to 10% of weight of scandium, yttrium, lanthanum, cerium, praseodymium, neodymium, promethium, samarium, europium, gadolinium, terbium, dysprosium, holmium, erbium, thulium and/or ytterbium

Furthermore metal salts of the aforementioned metals are particularly preferred. Such metal salts contain preferably at least one of the following metal ions: Li⁺, Be²⁺, Na⁺, Mg²⁺, K⁺, Ca²⁺, Sc³⁺, Ti²⁺, Ti⁴⁺, V²⁺, V³⁺, V⁴⁺, V⁵⁺, Cr²⁺, Cr³⁺, Cr⁴⁺, Cr⁶⁺, Mn²⁺, Mn³⁺, Mn⁴⁺, Mn⁵⁺, Mn⁶⁺, Mn⁷⁺, Fe²⁺, Fe³⁺, Co²⁺, Co³⁺, Ni²⁺, Cu⁺, Cu²⁺, Zn²⁺, Ga⁺, Ga³⁺, Al³⁺, Si⁴⁺, Y³⁺, Zr²⁺, Zr⁴⁺, Nb²⁺, Nb⁴⁺, Nb⁵⁺, Mo⁴⁺, Mo⁶⁺, Tc²⁺, Tc³⁺, Tc⁴⁺, Tc⁵⁺, Tc⁶⁺, Tc⁷⁺, Ru³⁺, Ru⁴⁺, Ru⁵⁺, Ru⁶⁺, Ru⁷⁺, Ru⁸⁺, Rh³⁺, Rh⁴⁺, Pd²⁺, Pd³⁺, Ag⁺, In⁺, In³⁺, Ta⁴⁺, Ta⁵⁺, W⁴⁺, W⁶⁺, Pt²⁺, Pt³⁺, Pt⁴⁺, Pt⁵⁺, Pt⁶⁺, Au⁺, Au³⁺, Au⁵⁺, Sn²⁺, Sn⁴⁺, Pb²⁺, Pb⁴⁺, La³⁺, Ce³⁺, Ce⁴⁺, Gd³⁺, Nd³⁺, Pr³⁺, Tb³⁺, Pr³⁺, Pm³⁺, Sm³⁺, Eu²⁺, Dy³⁺, Ho³⁺, Er³⁺, Tm³⁺, Yb³⁺.

Anions used include halogens such as F⁻, Cl⁻, Br⁻, oxides and hydroxides such as OH⁻, O²⁻, sulfates, carbonates, oxalates, phosphates such as HSO₄⁻, SO₄²⁻, HCO₃⁻, CO₃²⁻, HC₂O₄⁻, C₂O₄²⁻, H₂PO₄⁻, HPO₄²⁻, PO₄³⁻, and especially carboxylates such as HCOO⁻, CH₃COO⁻, C₂H₅COO⁻, C₃H₇COO⁻, C₄H₉COO⁻, C₅H₁₁COO⁻, C₆H₁₃COO⁻, C₇H₁₅COO⁻, C₈H₁₇COO⁻, C₉H₁₉COO⁻, PhCOO⁻, PhCH₂COO⁻.

Furthermore, salts of the following acids are preferred: sulfuric acid, sulfonic acid, phosphoric acid, nitric acid, nitrous acid, perchloric acid, hydrobromic acid, hydrochloric acid, formic acid, acetic acid, propionic acid, succinic acid, oxalic acid, gluconic acid, (glyconic acid, dextronic acid), lactic acid, malic acid, tartaric acid, tartronic acid (hydroxymalonic acid, hydroxypropanedioic acid), fumaric acid, citric acid, ascorbic acid, maleic acid, malonic acid, hydroxymaleic acid, pyruvic acid, phenylacetic acid, (o-, m-, p-) toluic acid, benzoic acid, p-aminobenzoic acid, p-hydroxybenzoic acid, salicylic acid, p-aminosalicylic acid, methanesulfonic acid, ethanesulfonic acid, hydroxyethanesulfonic acid, ethylenesulfonic acid, p-toluenesulfonic acid, naphthylsulfonic acid, naphthylaminesulfonic acid, sulfanilic acid, camphorsulfonic acid, china acid, quinic acid, o-methyl-mandelic acid, hydrogen-benzenesulfonic acid, methionine, tryptophan, lysine, arginine, picric acid (2,4,6-trinitrophenol), adipic acid, d-o-tolyltartaric acid, glutaric acid.

Furthermore, salts of amino acids containing for example one or more of the following amino acids are preferred: glycine, alanine, valine, leucine, isoleucine, serine, threonine, phenylalanine, tyrosine, tryptophan, lysine, arginine, histidine, aspartate, glutamate, asparagine, glutamine, cysteine, methionine, proline, 4-hydroxyproline, N,N,N-trimethyllysine, 3-methylhistidine, 5-hydroxylysine, O-phosphoserine, γ-carboxyglutamate, ε-N-acetyllysine, ω-N-methylarginine, citrulline, ornithine. Normally, amino acids having L-configuration are used. In another preferred embodiment at least some of the amino acids used have D-configuration.

Other preferred resorbable substances for the preparation of the implant are metal salts such as calcium chloride, calcium sulfate, calcium phosphate, calcium citrate, zinc chloride, zinc sulfate, zinc oxide, zinc citrate, iron sulfate, iron phosphate, iron chloride, iron oxide, zinc, magnesium chloride, magnesium sulfate, magnesium phosphate or magnesium citrate. Such metal salts are preferably used in amounts of 0.01-12% by weight.

Another preferred embodiment is the combination of resorbable metal or resorbable salt or a resorbable metal alloy together with a resorbable polymer. Such a combination may mean that the implant was produced of a mixture containing metal, metal alloy and/or metal salt together with a resorbable polymer. Such a combination may mean that the implant was produced from a mixture containing metal, metal alloy and/or metal salt and a biodegradable polymer, or that the implant is built from different layers, wherein one layer contains prevalently or exclusively the metal, metal salt and/or metal alloy, and the other or several other layers consist of one or more bioresorbable polymers.

The following biodegradable polymers are particularly suitable for the production of the bioresorbable outer wrapper. These resorbable polymers, however, may be added to the metal, metal salt or metal alloy building the inner structure, wherein the percentage of weight of organic polymers should not exceed 50% of weight of the overall inner structure, preferred be less than 40% by weight, more preferred less than 30% by weight and particularly preferred less than 20% by weight.

The following polymers may be used as resorbable or biodegradable polymers: polydioxanone, polycaprolactone, polygluconate, poly(lactic acid) polyethylene oxide copolymer, modified cellulose, polyhydroxybutyrate, polyamino acids, polyphosphate ester, polyvalerolactone, poly-ε-decalactone, polylactonic acid, polyglycolic acid, polylactides, polyglycolides, copolymers of the polylactides and polyglycolides, poly_ε-caprolactone, polyhydroxybutyric acid, polyhydroxybutyrates, polyhydroxyvalerates, polyhydroxybutyrate-co-valerate, poly(1,4-dioxane-2,3-one), poly(1,3-dioxane-2-one), poly-para-dioxanone, polyanhydrides, polymaleic acid anhydrides, polyhydroxy methacrylates, fibrin, polycyanoacrylate, polycaprolactone dimethylacrylates, poly-β-maleic acid, polycaprolactone butyl acrylates, multiblock polymers from oligocaprolactonediols and oligodioxanonediols, polyether ester multiblock polymers from PEG and poly(butylene terephthalates), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate), polyorthoesters, polyglycolic acid trimethyl carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide, soft polyurethanes, polyurethanes having amino acid residues in the backbone, polyetheresters such as polyethylene oxide, polyalkene oxalates, polyorthoesters as well as copolymers thereof, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivates thereof, heparins, chondroitin sulfate, dextran, β-cyclodextrins, copolymers with PEG and polypropylene glycol, gum arabic, guar, gelatin, collagen N-hydroxysuccinimide, lipids, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylenes, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, EPDM gums, silicones such as polysiloxanes, polydimethylsiloxanes, polyvinyl halogens and copolymers, cellulose ethers, cellulose triacetates, chitosans and copolymers and/or mixtures of the aforementioned polymers.

Particularly preferred biodegradable polymers are polydioxanone, polycaprolactone, polygluconate, polyamides, poly(lactic acid) polyethylene oxide copolymer, polysaccharides such as hyaluronic acid, chitosan, regenerated cellulose, modified cellulose, hydroxypropyl methylcellulose, collagen, gelatine, polyhydroxybutyrate (PHBT) and copolymers of polyhydroxybutyrate, polyanhydrides (PAN), polyphosphoesters, polyester, polyamino acids, polyglycolic acid, poly-ε-caprolactone, polyphosphate ester, polyorthoesters, poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polyethylene oxide (PEO), polydioxanone (PDS), polypropylene fumarate, poly(ethylglutamate-co-glutaminic acid), poly(tert-butyloxy-carbonylmethylglutamate), polycaprolactone (PCL), polycaprolactone-co-butylacrylate, polyphosphazene, poly(D,L-lactide-co-caprolactone) (PLA/PCL), poly(glycolide-co-caprolactone) (PGA/PCL), Maleinic acid anhydride and copolymers thereof, polyamino acids, polydepsipeptides, maleinic acid anhydride-copolymers, polyphosphazenes, polyiminocarbonates, poly[(97.5% dimethyltrimethylene carbonate)-co-(2.5% trimethylene carbonate)], cyanoacrylate, polyethylene oxide as well as copolymers and mixtures of the aforementioned polymers.

Further preferred are polyunsaturated fatty acids cross-linking via autopolymerization such as eicosapentaenoic acid, timnodonic acid, docosahexaenoic acid, arachidonic acid, linoleic acid, α-linolenic acid, γ-linolenic acid as well as mixtures of the aforementioned fatty acids, and especially mixtures of pure unsaturated compounds. Oils such as linseed oil, hempseed oil, corn oil, walnut oil, rape oil, soy bean oil, sun flower oil, poppy-seed oil, safflower oil, wheat germ oil, safflor oil, grape-seed oil, evening primrose oil, borage oil, black cumin oil, algae oil, fish oil, cod-liver oil also contain a high amount of unsaturated fatty acids and thus can be used too.

Further preferred substances for the polymeric coating are omega-3- and omega-6 fatty acids as well as all substances which at least carry one omega-3- and/or omega-6 fatty acid residue. Such substances are well enabled for autopolymerization.

The ability of curing, i.e. the ability of autopolymerization, lies in the composition of the oils, also named drying oils, and is based in the high content of essential fatty acids, precisely in the double bonds of the unsaturated fatty acids. In the air radicals are built through oxygen at the double bonding sites of the fatty acid molecules that initiate radical polymerization and propagate so that the fatty acids cross-link among themselves, thereby losing their double bonds.

Autopolymerization is also named self-polymerization and can for example be initiated through oxygen, especially oxygen from the air, or other radical formers. Another option consists in the initiation of the autopolymerization through electromagnetic radiation, especially light.

Further preferred resorbable polymers are polymethyl methacrylates (PMMA), polytetrafluoroethylene (PTFE), polyurethanes, polyvinyl chlorides (PVC), polydimethylsiloxanes (PDMS), polyesters, nylons and polylactides and polyglycolides.

Particularly preferred for the production of the outer polymeric wrapper are polyesters, polylactides as well as copolymers of diols and esters or diols and lactides. For example, ethane-1,2-diol, propane-1,2-diol or butane-1,2-diol can be used as diols.

Especially polyesters are used for the polymeric layer. From the group of polyesters such polymers are preferred which have the following repetitive unit:

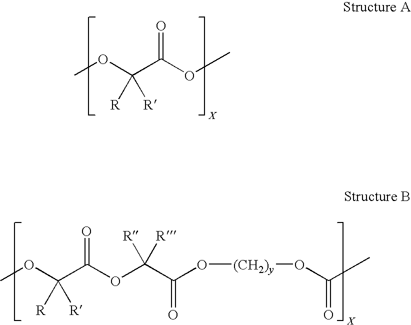

In the depicted repetitive units R, R′, R″ and R′″ stand for an alkyl residue of 1 to 5 carbon atoms, especially methyl, ethyl, propyl, isopropyl, n-butyl, s-butyl, t-butyl, iso-butyl, n-pentyl or cyclopentyl and preferably methyl or ethyl. Y stands for an integer of 1 to 9 and X stands for the polymerization degree. Particularly preferred are the following polymers with the shown repetitive units:

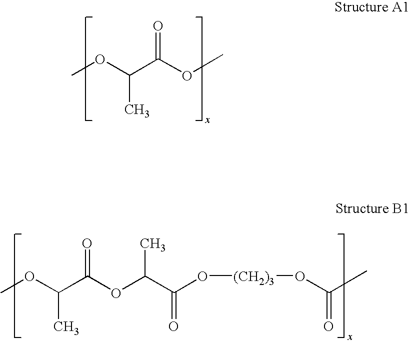

These resorbable polymers are prepared on the basis of lactic and glycolic acid. Basically the use of resorbable polymers is particularly preferred. Homopolymers of lactic acid (polylactides) are mostly used in the production of resorbable medical implants. Copolymers of lactic and glycolic acid can be used as raw materials in the production of capsules with an active agent for the controlled release of pharmaceutical active agents.

Thus particularly polymers on the basis of lactic and glycolic acid and copolymers (alternating or static) and block copolymers (e.g. triblock copolymers) of both acids are preferred.

Further representatives of resorbable polymers shall be those bioresorbable polymers named Resomers® from Boehringer Ingelheim GmbH, namely poly(L-lactide)s with the general formula —(C6H8O4)n- such as L 210, L 210 S, L 207 S, L 209 S, the poly(L-lactide-co-D,L-lactide)s with the general formula —(C6H8O4)n- such as LR 706, LR 708, L 214 S, LR 704, the poly(L-lactide-co-trimethyl carbonate)s with the general formula —[(C6H8O4)x-(C4H6O3)y]n-such as LT 706, the poly(L-lactide-co-glycolide)s with the general formula —[(C6H8O4)x-(C4H4O4)y]n- such as LG 824, LG 857, the poly(L-lactide-co-ε-caprolactone)s with the general formula —[(C6H8O4)x-(C6H10O2)y]n- such as LC 703, the poly(D,L-lactide-co-glycolide)s with the general formula —[(C6H8O4)x-(C4H4O4)y]n- such as RG 509 S, RG 502H, RG 503H, RG 504H, RG 502, RG 503, RG 504, the poly(D,L-lactide)s with the general formula —(C6H8O4)n- such as R 202 S, R 202H, R 203 S and R 203H. Resomer® 203 S is herein the successor of the particularly preferred polymer Resomer® R 203. Particularly preferred is the use of R203 and LT 706 in a weight ratio of 70% to 30% of weight.

It shall be mentioned again that the embodiments described herein are not providing a bioresorbable stent or providing a bioresorbable metal alloy for a stent but the combination of a bioresorbable stent scaffold with a polymeric bioresorbable coating that enables the entry of water and the escape of ions, and in the particularly preferred embodiments the inner stent scaffold is significantly faster degraded than the outer coating.

Metals and metal alloys suitable for the production of biodegradable stent scaffolds are sufficiently known from the literature. Basically any metal alloy containing as major component magnesium, zinc, calcium or iron can be used.

An embodiment comprises applying a polymeric coating onto biodegradable metal scaffolds wherein the polymeric coating releases the degradation products of the inner stent scaffold to the surrounding, i.e. they can pass out through the polymeric coating, and in a preferred embodiment the polymeric coating starts dissolving not before the inner stent scaffold is already substantially biodegraded. This means that that the polymeric coating encases safely the inner stent scaffold or the fragments of the inner stent scaffold for so long until the stent has grown into the surrounding tissue or no fragments of the inner stent scaffold that may cause heart infarction can pass anymore the polymeric coating. This problem can be solved by a plurality of embodiments wherein the skilled person knows the biodegradable stent materials, respectively metal alloys, as well as the biodegradable polymeric coating and wherein they have to be combined only according to the teachings herein. When the person skilled in the art knows the teaching described herein such combinations are not inventive anymore but only require some standard tests for determining the ion permeability and the degradation velocity of the stent scaffold and the polymeric coating.

For example, the ion permeability can be determined by placing a coated stent in an aqueous solution and measuring the electric conductivity of the solution after certain time intervals, or by determining the osmotic pressure, or by determining the ion content of the solution by means of spectroscopic methods.

A particularly preferred embodiment is directed to implants with an inner metal structure which is coated with a biodegradable polymer selected from polymethyl methacrylate (PMMA), polytetrafluoroethylene (PTFE), polyurethane, polyvinyl chloride (PVC), polydimethylsiloxane (PDMS), polyester, nylon or polylactide, and particularly with a polyester and/or polylactide. The polymeric coating further displays holes, openings or channels which run perpendicular to the longitudinal axis of the respective stent strut.

The pores, holes, openings or channels are preferably evenly distributed over the stent surface and substantially run perpendicular through the polymer towards the inner metal scaffold. Preferably, there are 1 to 20 such pores, holes, openings or channels per mm² surface of the stent strut.

The complete stent surface, i.e. the surface of the polymeric wrapper, as well as the pores, holes, openings or channels, or a part of the stent surface and a part of the pores, holes, openings or channels, or only a part of the pores, holes, openings or channels can be filled with an active agent or a composition containing at least one active agent.

The polymeric coating is applied by known procedures such as the spray method, dipping method, plasma method, brush method, squirting method, electrospinning method or pipetting method onto the structure of the basic scaffold and preferably adheres to it. In general, the pores, holes, openings or channels are applied into the coating only after the coating procedure by means of a laser, temperature, mechanic contact or chemical influence wherein the generation of the pores, holes, openings or channels is relatively simple with a laser, but is not the most suitable method for all types of polymers.

Further advantageous embodiments comprise resorbable implants containing at least one pharmacologically active substance in the biodegradable layer and optionally on the biodegradable layer. Preferred pharmacologically active substances are antiproliferative, antimigrative, antiangiogenic, anti-inflammatory, antiphlogistic, cytostatic, cytotoxic and/or antithrombogenic active agents, antirestenotic active agents, corticoids, sexual hormones, statins, epothilones, prostacyclins, angiogenesis inductors. Among these substances antiproliferative, anti-inflammatory, antineoplastic, antimigrative, antiphlogistic, cytostatic, cytotoxic and/or antithrombogenic agents and antirestenotic agents are preferred.

Examples for anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombotic agents are: abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, bombrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a, 4-hydrorxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, inhibitor 2ω of smc proliferation, mitoxanthrone, mycophenolate c-myc antisense, b-myc antisense, β-lapachone, podophyllotoxin, podophyllic acid 2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, NFkB, angiopeptine, monoclonal antibodies inhibiting muscle cell proliferation, bFGF antagonists, probucol, prostaglandins, 1-hydroxy-11-methoxycanthin-6-one, scopoletin, NO donors such as pentaerythritol tetranitrate and sydnonimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof such as 6-α-hydroxy-paclitaxel, taxoteres, carbon suboxides (MCS) and macrocylic oligomers thereof, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF 1, active agents from the group of antibiotics such as cefadroxil, cefazolin, cefaclor, cefoxitin, tobramycin, gentamicin, penicillins such as dicloxacillin, oxacillin, sulfonamides, metronidazole, enoxaparin, desulfated and N-reacetylated heparin, tissue plasminogen activator, GpIIb/IIIa platelet membrane receptor, antibodies to factor Xa inhibitor, heparin, hirudin, r-hirudin, PPACK, protamine, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators such as p65, NF-kB or Bcl-xL antisense oligonucleotides, halofuginone, nifedipine, tocopherol, tranilast, molsidomine, tea polyphenols, epicatechin gallate, epigallocatechin gallate, leflunomide, etanercept, sulfasalazine, etoposide, dicloxacylline, tetracycline, triamcinolone, mutamycin, procainimide, retinoic acid, quinidine, disopyrimide, flecamide, propafenone, sotalol, natural and synthetically obtained steroids such as inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, clotrimazole, flucytosine, griseofulvin, ketoconazole, miconazole, nystatin, terbinafine, antiprotozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceantinoside C, yadanziosides N and P, isodeoxyelephantopin, tomenphantopin A and B, coronarin A, B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, maquiroside A, marchantin A, maytansin, lycoridicin, margetine, pancratistatin, liriodenine, bisparthenolidine, oxoushinsunine, periplocoside A, ursolic acid, deoxypsorospermin, psychorubin, ricin A, sanguinarine, manwu wheat acid, methylsorbifolin, chromones of spathelia, stizophyllin, mansonine, strebloside, dihydrousambaraensine, hydroxyusambarine, strychnopentamine, strychnophylline, usambarine, usambarensine, liriodenine, oxoushinsunine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

Preferred active agents are paclitaxel and its derivatives such as 6-α-hydroxy-paclitaxel or baccatine and other taxoteres, sirolimus, everolimus, biolimus A9, pimecrolimus, zotarolimus, tacrolimus, erythromycine, midecamycine, josamycine and triazolopyrimidine.

Particularly preferred are paclitaxel (Taxol®) and all derivatives of paclitaxel such as 6-α-hydroxy-paclitaxel and sirolimus and their derivatives.

The resorbable implants are preferably supporting prostheses for channel-like structures, and in particular stents for blood vessels, the urinary tract, the airways, oesophagus, bile ducts or the intestinal tract.

Among these stents, stents for blood vessels, or in general for the cardiovascular system, are preferred.

In general, these stents are self-expandable or balloon-expandable containing preferably at least one anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent preferably in the polymeric coating and/or the holes, openings, pores and/or channels.

In general, the biodegradable layer serves as a carrier for the at least one anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent. This agent prevents inflammation which may be caused by the stent and regulates the growth of mainly smooth muscle cells (respectively coronary endothelial cells) on the stent. The stent allows for a regeneration of the supporting tissue or the supporting vessel section. When the tissue has regenerated it is able to support the vessel by itself and doesn't require anymore an additional support through the stent. At this stage the stent having grown into the vascular wall is already considerably degraded and in general the inner structure doesn't exist anymore. The degradation process continues until the stent is completely dissolved without disintegrating into solid fragments which could move freely in the bloodstream.

The terms “resorbable” or “degradable” or “biodegradable” refer to the fact that the human or animal body is able to slowly dissolve the implant to components which are present in blood or solved in other body fluids.

The preferred stents are designed in a grate-like shape wherein the individual struts of the grate structure have similar cross sectional areas. A ratio of less than 2 is preferred for the largest and the smallest cross sectional area. The similar cross sectional areas of the struts lead to an equal degradation of the stent.

Furthermore it is preferred that the ring-shaped bars are linked through connecting bars wherein the connecting bars preferentially display a smaller cross sectional area or a smaller minimal diameter than the bars forming the ring-shaped bars. This leads to a faster degradation of the connecting bars in the human or animal body in comparison to the ring-shaped bars. Thus the axial flexibility of the stent is augmented faster than the supporting capacity of the stent decreases as a consequence of the degradation of the ring-shaped bars.

The medical implant, especially the stent, can be coated by a spray, pipetting, brush, squirting, plasma elimination, dipping, electrospinning or “soap bubble” method wherein a polymer is dissolved in a solvent and the solvent is applied onto the implant.

The polymer may also be preformed in a tube-like form and applied onto the outer or inner surface of the stent.

Suitable solvents include water and preferably organic solvents such as chloroform, methylene chloride (dichloromethane), acetone, tetrahydrofuran (THF), diethyl ether, methanol, ethanol, propanol, isopropanol, diethyl ketone, dimethylformamide (DMF), dimethylacetamide, acetic acid methyl ester, acetic acid ethyl ester, dimethyl sulfoxide (DMSO), benzene, toluene, xylene, t-butyl methyl ether (MTBE), petroleum ether (PE), cyclohexane, pentane, hexane, heptane, wherein chloroform and acetic acid ethyl ester are particularly preferred.

The at least one active agent to be applied can be dissolved, emulated, suspended or dispersed in a suitable solvent or even together with the polymer. Potential substances to be applied include the pharmacologically active agents mentioned above and the polymers described above.

The polymeric coating should be relatively equal and should have a thickness of 0.01 to 10 μm. The desired layer thickness depends also from the respective polymer and can be realized in several coating steps.

### EXAMPLES

### Example 1

A stent consists of:

| | |
|---|---|
| 90% (w/w) | of zinc |
| 6% (w/w) | of magnesium |
| 1% (w/w) | of calcium |
| 2% (w/w) | of yttrium |
| 1% (w/w) | of other metals, metal salts, non-metals, carbon, sulphur, |
| | nitrogen, oxygen, hydrogen. |

The stent according to example 1 is coated in a dipping process with a solution of a polyglycol and doxorubicin. Upon drying, the dipping process is repeated another two times.

### Example 2

A stent consists of:

| | |
|---|---|
| 46% (w/w) | of zinc |
| 46% (w/w) | of magnesium |
| 6% (w/w) | of yttrium |
| 2% (w/w) | of other metals, metal salts, non-metals, carbon, sulphur, |
| | nitrogen, oxygen, hydrogen. |

The stent according to example 2 is coated in a spraying process at intervals with a solution of a polylactide and the active agent paclitaxel in chloroform. Upon drying, the polymeric coating is fused at discrete spots by means of a temperature transmitter in order to form holes. Then the holes are filled with a solution of paclitaxel in DMSO and dried.

### Example 3

A stent consists of:

| | |
|---|---|
| 75% (w/w) | of zinc |
| 15% (w/w) | of calcium |
| 4% (w/w) | of yttrium |
| 0.7% (w/w) | of manganese |
| 0.8% (w/w) | of iron |
| 4.5% (w/w) | of other metals, metal salts, non-metals, carbon, sulphur, |
| | nitrogen, oxygen, hydrogen. |

The stent according to example 3 is coated in a spraying process at intervals with a solution of a polygluconate in methylene chloride. Upon drying, the polymeric coating is fused at discrete spots by means of acid treatment in order to form holes. Upon thorough removal of possibly remaining acid through several washes and dryings of the stent the holes are filled by means of a pipette with an ethanolic solution containing 30% by weight of paclitaxel and the contrast medium iopromide. Subsequently, drying occurs under soft airflow at room temperature.

### Example 4

A stent consists of:

| | |
|---|---|
| 29% (w/w) | of iron |
| 13% (w/w) | of calcium |
| 53% (w/w) | of magnesium |
| 3% (w/w) | of yttrium |
| 0.2% (w/w) | of manganese |
| 0.8% (w/w) | of iron |
| 1.0% (w/w) | of other metals, metal salts, non-metals, carbon, sulphur, |
| | nitrogen, oxygen, hydrogen. |

The stent according to example 4 is coated in a spraying process at intervals with a solution of a polyanhydride and rapamycin in chloroform. Upon drying, a laser cuts channels along the struts into the polymeric coating. Rapamycin in and a fatty acid ester such as isopropyl palmitate are sprayed onto the stent surface until a concentration of the active agent of 3 μg rapamycin per mm² stent surface results.

### Example 5

A stent consists of:

| | |
|---|---|
| 7% (w/w) | of iron |
| 72% (w/w) | of calcium |
| 16% (w/w) | of zinc |
| 1.1% (w/w) | of yttrium |
| 3.9% (w/w) | of other metals, metal salts, non-metals, carbon, sulphur, |
| | nitrogen, oxygen, hydrogen. |

The stent according to example 5 is coated in a spraying process at intervals with a solution of poly-ε-caprolactone in methylene chloride. Upon drying, the polymeric coating is roughened by means of a sandblast so that holes, channels and opening up to the metal inner scaffold are formed. Then the holes are filled with a solution of simvastatin in acetone by means of the pipetting method.

### Example 6

A stent consists of:

| | |
|---|---|
| 5% (w/w) | of iron |
| 40% (w/w) | of calcium |
| 40% (w/w) | of magnesium |
| 5% (w/w) | of zinc |
| 0.3% (w/w) | of yttrium |
| 9.7% (w/w) | of other metals, metal salts, non-metals, carbon, sulphur, |
| | nitrogen, oxygen, hydrogen. |

The stent according to example 6 is coated in a spraying process at intervals with a solution of a polyurethane and trapidil in methylene chloride. Upon drying, the polymeric coating is roughened by means of a sandblast so that holes, channels and opening up to the metal inner scaffold are formed. Then the entire stent surface is sprayed two times with a solution of paclitaxel in methanol and dried after each spraying step.

### Example 7

A stent consists of

| | |
|---|---|
| 5% (w/w) | of sodium |
| 44% (w/w) | of calcium |
| 44% (w/w) | of magnesium |
| 1% (w/w) | of zinc |
| 1% (w/w) | of tantalum |
| 0.5% (w/w) | of yttrium |
| 4.5% (w/w) | of other metals, metal salts, non-metals, carbon, sulphur, |
| | nitrogen, oxygen, hydrogen. |

The stent according to example 6 is coated in a brushing process with a viscous solution of hydroxymethyl cellulose and 2-methylthiazolidine-2,4-dicarboxic acid in methanol. Upon drying, by means of ion bombardment micropores are generated that reach up to the metal inner scaffold. Then the entire stent surface is sprayed two times with paclitaxel solved in chloroform and dried after each spraying step.

### Example 8

### Determination of the Elution Behaviour of Paclitaxel in PBS Buffer

2 ml of PBS buffer are added to one stent respectively in a sufficiently small container, sealed with Parafilm and incubated in a compartment drier at 37° C. After each chosen time interval the supernatant is pipetted off and its UV absorption is measured at 306 nm.

### Example 9

### Biocompatible Coating of a Biodegradable Stent with Linseed Oil and α-Linolenic Acid

After cleaning the stents with acetone and ethanol a mixture of 0.2% linseed oil and 0.5% α-linolenic acid dissolved in ethanol is produced and evenly sprayed onto the stent. The stent will be at a temperature of

### Example 10

### Biocompatible Coating of a Biodegradable Stent with Linseed Oil and the Synthetic Polymer Polyvinyl Pyrrolidone (PVP) in a Two-Layer System Under Addition of a Restenosis-Inhibiting Agent

After cleaning the stents a first layer of 0.35% by weight rapamycin solved in chloroform is sprayed onto the stent. Upon drying of this layer at room temperature, a second layer of a chloroform solution of 0.25% linseed oil and 0.1% PVO is sprayed upon.

### Example 11

### Covalent Hemocompatible Coating of Biodegradable Stents

### Example 11a

### Preparation of Desulfated Reacetylated Heparin

100 ml of Amberlite IR-122 cation-exchange resin were filled in a column of 2 cm diameter, converted with 400 ml of 3M HCl into the H⁺ form and washed with aqua dest. until the eluate was free of chloride and the pH was neutral. 1 g of sodium heparin was dissolved in 10 ml water, given on the cation-exchange column and eluted with 400 ml water. The eluate was dropped into a receiver with 0.7 g pyridine and subsequently titrated with pyridine up to pH 6 and freeze-dried. 90 ml of a 6/3/1 mixture of DMSO/1,4-dioxane/methanol (v/v/v) were added to 0.9 g of heparin pyridinium salt in a round-bottom flask with a reflux condenser and heated to 90° C. for 24 hours. Then 823 mg of pyridinium chloride were added and heated to 90° C. for another 70 hours. Subsequently it was diluted with 100 ml water and titrated up to pH 9 in diluted sodium hydroxide. The desulfated heparin was dialyzed against water and freeze-dried.

100 mg of the desulfated heparin were dissolved in 10 ml water, cooled to 0° C. and 1.5 ml methanol were added under stirring. To this solution 4 ml Dowex 1×4 anion-exchange resin in the Off form and subsequently 150 μl acetic acid anhydride were added and stirred for 2 hours at 4° C. Thereafter the resin is filtered off and the solution is dialyzed against water and freeze-dried.

### Example 11b

### Covalent Coating of the Stents

The stents are degreased in an ultrasonic bath with acetone and ethanol for 15 minutes and dried in a compartment drier at 100° C. Then they were dipped into a 2% solution of 3-aminopropyltrieethoxysilane in a mixture of ethanol/water (50/50: (v/v)) for 5 minutes and subsequently dried at 100° C. for 5 minutes. After that the stents were washed overnight with demineralised water.

3 mg of desulfated and reacetylated heparin were solved in 30 ml of 0.1 M MES buffer (2-(N-morpholino)ethansulfonic acid) at 4° C. and pH 4.75 and 30 mg of N-cyclohexyl-N′-(2-morpholinoethyl)carbodiimide-methyl-p-toluene sulfonate were added. 10 stents were stirred in this solution for 15 hours at 4° C. Subsequently it was washed with water, 4 M NaCl solution and water, for 2 hours each. The stents were extensively dried in airflow and in a vacuum exsiccator and stored.

### Example 12

### Coating of a Biodegradable Stent with an Active Agent in the Cavities of the Entirely Covering Matrix Made of a Biodegradable Polymer

a) Coating of the Stents with a Pure Matrix in a Spray Process

### Preparation of the Spraying Solution:

176 mg of PLGA were weighed and filled up with chloroform to 20 g. The stents were sprayed each with 3 ml of the spaying solution and then dried overnight.
or
Coating with a Matrix Loaded with an Active Agent
spraying solution: a PLGA/taxol solution of 145.2 mg PLGA and 48.4 mg taxol are filled up with chloroform to 22 g. The stents were sprayed each with 3 ml of the spaying solution and then dried overnight.

### b) Preparation of the Cavities in the Pipetting Mode

After complete drying of the polymer-coated stents cavities are generated in the abluminal surface of the stent by spot selective etching of the polymeric layer with a defined amount of chloroform or another suitable solvent in such a way that the cavities are evenly distributed over the stent struts of the entire stent body. Possibly remaining solvent is removed in the airstream immediately after the generation of each cavity.

c) Coating of the Cavities with Hydrophilic Polymers Loaded with an Active Agent in the Pipetting Mode

By means of the pipetting mode the generated cavities are filled with a viscous solution loaded with an active agent. The solution must be so viscous that it can't flow out of the cavity, respectively the solvent evaporates so rapidly that the solution hardens and the surrounding matrix is not dissolved.

For example, a rapamycin/PVP solution can be used wherein the content of rapamycin in the solution amounts to 35%. In combination with one or more active agents the content of rapamycin shouldn't be less than 20%.

The thus filled coated stent is dried afterwards.

### Example 13

### Coating of the Cavities with Pure Active Agent in the Pipetting Mode

Therefore 8.8 mg taxol are filled with chloroform to 2 g and pipetted into the cavities.

### Example 14

### Coating of the Cavities with an Active Agent and a Substance which Accelerates Membrane Permeability in the Pipetting Mode

Therefore 450 μl of ethanol are mixed with 100 μl isopropyl myristate. This solution is added to a solution of 4.5 ml acetone and 150 mg epothilone A.

Subsequently the cavities are filled by means of the pipetting method and dried.

### Example 15

### Complete Coating of a Cylindrical Stent Body

a. Precoating of Stents in a Spray Process

A stent according to example 1 to 7 is fixed to the rod of a rotator and is sprayed with a 1% polyurethane solution at a low rotational speed with very slow up and down movements of the spray gun.

b. Entire Coating of a Sprayed Stent by Dip Coating

Polyurethane is dissolved in THF so that a 14% solution results. A stent precoated according to example 15a is cautiously moved on a fitting moulding tool.

The tool with the mounted stent is first predipped into pure THF for a short time. Then it is slowly dipped into the 14% urethane solution. After 15 seconds the tool with the stent is slowly pulled out again and rotated further so that the PU is evenly distributed on the stent and dried. When it doesn't peter out anymore the core is dried under the exhaust hood and subsequently tempered in the compartment drier at 95° C. After cooling the stent including the PU wrapper is very cautiously removed from the tool. It must be taken care that the PU wrapper doesn't get any cracks or holes. The cleaning of the stents entirely coated in such a way is done very thoroughly under lukewarm water flow.

### Example 16

### Coating of the Entire Surface of a Sprayed Stent with PU/Terguride in the Dipping Mode

The dipping solution consists of 30% by weight terguride in polymer which then is diluted to 10% in THF. The further handling is done as in example 15b.

### Example 17

### Partial Coating of a Biodegradable Stent (D=3 mm)

Solution: 3.2 mg of PU solved in 20 ml N-methyl-2-pyrrolidone

A spray-coated stent is moved onto a fitting freely revolvable moulding tool so that it rests completely on the smooth basement.

The application of the coating is realized in at least two steps wherein solution is picked up in a brush hair and is applied onto the area to be coated until the area is completely covered with solution.

When every selected area to be coated is filled in the desired coating thickness the stent is dried at 90° C. After cooling the stent is removed from the moulding tool.

### Example 18

A bioresorbable stent of the following composition is prepared according to EP 1419793 B1:

| | |
|---|---|
| magnesium | 91% (w/w) |
| yttrium | 4% (w/w) |
| neodymium | 4% (w/w) |
| other | 1% (w/w) |

“other” are: other metals, metal salts, non-metals, carbon, sulphur, nitrogen, oxygen, hydrogen.

This magnesium stent was provided with a bioresorbable coating of PLLA/PGA and the degradation velocity of the uncoated stent, of the polymeric coating of PLLA/PGA on a stainless steel stent as well as of the coated stent was determined according to example 18.

The uncoated magnesium stent was dissolved completely in PBS buffer (phosphate buffer with 14.24 g NaH₂PO₄, 2.72 g K₂HPO₄ and 9 g NaCl; pH 7.4; T=37° C.) during 10 days, while the coated stent according to example 18 dissolved completely inside the coating during ca. 12 days.

The PLLA/PGA coating on the stainless steel stent as well as on the stent according to example 18 were dissolved in PBS buffer (phosphate buffer with 14.24 g NaH₂PO₄, 2.72 g K₂HPO₄ and 9 g NaCl; pH 7.4; T=37° C.) after ca. 6-8 weeks.

An optical analysis of a stent according to example 18 after 10, 20, 30 and 40 days in PBS buffer shows that already after 20 days the inner stent scaffold is completely dissolved and washed out off the polymeric coating whereas PLLA/PGA didn't display yet significant dissolution and at least no visible holes.

### Example 19

### Coating of Biodegradable Stents on the Luminal and Abluminal Side with Two Polylactides (PLGA 75/25 and PLGA 50/50) which Degrade at a Different Velocity

The biodegradable stent according to example 1 to 7 is hung horizontally on a thin metal rod (d=0.2 mm) which is mounted on the rotational axis of a rotation and advance device so that the inner side of the stent does not contact the rod. While rotating slowly about its longitudinal axis the faster degradable polymer (PLGA 50/50) dissolved in chloroform is applied onto the stent struts on the abluminal surface of the stent using the continuous pipetting mode (optionally, brushing mode, ink jet mode, ballpoint mode). Drying occurs under soft airflow at room temperature.

The abluminally coated stent is now coated from the luminal side with a slower degradable polylactide (PLGA 75/25). Therefore the stents are brushed along the struts with the polymeric solution by means of a brush hair. Afterwards drying occurs again under soft airflow at room temperature.

Polymeric solution: 176 mg of polylactide is weighed and filled to 20 g with chloroform.

Optionally, the active agents or combinations of active agents can be mixed into the polymeric solution. For example, an anti-inflammatory, antiproliferative agent such as rapamycin is very suitable for the abluminal side which faces the vascular wall, while an antithrombogenic agent on the luminal surface which is exposed to the bloodstream ensures the necessary thrombosis prophylaxis, for example:
a) abluminal side: polylactide PLGA 50/50 and rapamycin (solution of 145.2 mg polylactide and 48.4 mg rapamycin is filled up to 22 g with chloroform).
b) luminal side: polylactide PLGA 75/25 and clopidogrel (solution of 145.2 mg polylactide and 48.4 mg clopidogrel is filled up to 22 g with chloroform).

### Example 20

### Biocompatible Coating of Biodegradable Stents with Linseed Oil and Paclitaxel

Linseed oil and paclitaxel (80:20) are solved in a ratio of 1:1 in chloroform and then sprayed on an evenly rotating stent. After evaporation of chloroform in soft airflow the stent is stored in a compartment drier at 80° C.

### Example 21

### Biocompatible Coating of Stents with Linseed Oil and Polyvinyl Pyrrolidone (PVP) in a Two-Layer System Under Addition of Paclitaxel

After cleaning the stents a first layer of 0.25% by weight paclitaxel solved in chloroform is sprayed onto the stent. Upon drying of this layer at room temperature the second layer of a chloroform solution with 0.25% linseed oil and 0.1% PVP is sprayed upon and dried overnight at 70° C.

### Example 22

### Measurement of the Degradation of the Bioresorbable Stent Material

### a) Preparation of the Alloy by Means of Powder Metallurgy

The single components are ground, well mixed and pressed under high pressure into the desired form and finally sintered. By this preparation compact and nearly sealed bodies can be generated under avoidance of the melting process (molten bath).

### b) Degradation Measurement

This work piece produced as a tube and weighed out to the fourth decimal place is placed in a suitable silicone tube, similar as a stent. The tube ends are located in a container filled with PBS buffer (phosphate buffer with 14.24 g NaH₂PO₄, 2.72 g KH₂PO₄ and 9 g NaCl; pH 7.4; T=37° C.). By means of a connected peristaltic pump the buffer is pumped from the container through the tube and released again into the container wherein a filter at the tube end ensures that possibly present particles are not pumped through the system. At the same time the filter serves as a first control for the undesired formation and detachment of larger particles during degradation of the material. After the end of the predetermined trial time the tube segment including the remaining material is cautiously cut out, taken out without loss, dried, weighed and described.

The time course of the degradation of an alloy is documented by carrying out several of the described experimental designs which are terminated after differentially set time intervals. In such a way the course of degradation can be obtained based on time-dependent weight loss (at physiological 37° C. and pH 7.4 in a not-static system).

### Example 23

### Measurement of Polymer Degradation in a Flow System

The degradation of biodegradable polymers is investigated under physiological conditions (pH 7.4; T=37° C.) in PBS buffer (phosphate buffer). Therefore the biodegradable polymer is first solved in a volatile solvent such as chloroform. Subsequently a thin lamina is cast and dried until constancy of weight under vacuum.

The exactly weighed lamina is brought into a so-called Baumgartner chamber modified according to Sakarassien [Sakarassien et al., J Lab-Clin. Med 102(4): 522 (1983)] (see the flatbed perfusion system) and PBS buffer is conducted over the laminar surface at a chosen flow velocity by means of a peristaltic pump. The experiment is carried out at different experimental times and the degradation behaviour is noted on the base of the laminar weight (after drying under vacuum until constancy of weight and the state respectively the changes of the polymer characteristics are noted.

### Example 24

A bioresorbable stent with the following composition is produced according to EP 1419793 B1:

| | |
|---|---|
| magnesium | 90% by weight |
| yttrium | 5% by weight |
| zirconium | 4% by weight |
| others | 1% by weight |

“others” are: other metals, metal salts, non-metals, carbon, sulphur, nitrogen, oxygen, hydrogen

This magnesium stent was provided with a bioresorbable coating of PGA/PTMC and the degradation velocity of the uncoated stent and of the polymeric coating of PGA/PTMC on a stainless steel stent as well as of the coated stent was determined according to Example 24.

The uncoated magnesium stent dissolved completely in PBS buffer (phosphate buffer with 14.24 g NaH₂PO₄, 2.72 g KH₂PO₄ and 9 g NaCl; pH 7.4; T=37° C.) during 13 days, while the coated stent according to Example 24 dissolved completely inside the coating during ca. 15 days.

The PGA/PTMC coating on the stainless steel stent as well as on the stent according to Example 24 was dissolved in PBS buffer (phosphate buffer with 14.24 g NaH₂PO₄, 2.72 g KH₂PO₄ and 9 g NaCl; pH 7.4; T=37° C.) after ca. 15-18 weeks.

An optical analysis of a stent according to Example 24 after 20 days and after 40 days in PBS buffer showed that already after 20 days the inner stent scaffold was completely dissolved and washed off the polymeric coating wherein the PLLA/PGA didn't display significant dissolution traits, at least no visible holes. The polymeric coating was thus still entirely intact while the inner stent scaffold had dissolved completely and the metal ions were released through the polymer to the buffer solution.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A biodegradable stent comprising an inner bioresorbable scaffold containing at least one metal and surrounded by a polymeric biodegradable coating.

2. Biodegradable stent according to claim 1, wherein the inner bioresorbable scaffold containing at least one metal is a metal, metal alloy, metal oxide, metal salt, metal carbide, metal nitride or a mixture of the aforementioned substances.

3. Biodegradable stent according to claim 1, wherein the inner bioresorbable scaffold containing at least one metal has a potential of −0.53 eV measured in comparison to the calomel electrode.

4. Biodegradable stent according to claim 1, wherein the inner bioresorbable scaffold containing at least one metal is faster biodegradable than the polymeric outer wrapper.

5. Biodegradable stent according to claim 1, wherein the inner bioresorbable scaffold containing at least one metal contains at least one metal selected from the group consisting of lithium, sodium, magnesium, aluminum, potassium, calcium, scandium, titanium, vanadium, chromium, manganese, iron, cobalt, nickel, copper, zinc, gallium, silicon, yttrium, zirconium, niobium, molybdenum, technetium, ruthenium, rhodium, palladium, silver, indium, tin, lanthanum, cerium, praseodymium, neodymium, promethium, samarium, europium, gadolinium, terbium, dysprosium, holmium, erbium, thulium, ytterbium, lutetium, tantalum, tungsten, rhenium, platinum, gold, lead.

6. Biodegradable stent according to claim 1, wherein the inner bioresorbable scaffold containing at least one metal contains at least one metal ion of the following oxidation stage: Li⁺, Na⁺, Mg²⁺, K⁺, Ca²⁺, Sc³⁺, Ti²⁺, Ti⁴⁺, V²⁺, V³⁺, V⁴⁺, V⁵⁺, Cr²⁺, Cr³⁺, Cr⁴⁺, Cr⁶⁺, Mn²⁺, Mn³⁺, Mn⁴⁺, Mn⁵⁺, Mn⁶⁺, Mn⁷⁺, Fe²⁺, Fe³⁺, Co²⁺, Co³⁺, Ni²⁺, Cu⁺, Cu²⁺, Zn²⁺, Ga⁺, Ga³⁺, Al³⁺, Si⁴⁺, Y³⁺, Zr²⁺, Zr⁴⁺, Nb²⁺, Nb⁴⁺, Nb⁵⁺, Mo⁴⁺, Mo⁶⁺, Tc²⁺, Tc³⁺, Tc⁴⁺, Tc⁵⁺, Tc⁶⁺, Tc⁷⁺, Ru³⁺, Ru⁴⁺, Ru⁵⁺, Ru⁶⁺, Ru⁷⁺, Ru⁸⁺, Rh³⁺, Rh⁴⁺, Pd²⁺, Pd³⁺, Ag⁺, In⁺, In³⁺, Ta⁴⁺, Ta⁵⁺, W⁴⁺, W⁶⁺, Pt²⁺, Pt³⁺, Pt⁴⁺, Pt⁵⁺, Pt⁶⁺, Au⁺, Au³⁺, Au⁵⁺, Sn²⁺, Sn⁴⁺, Pb²⁺, Pb⁴⁺, La³⁺, Ce³⁺, Ce⁴⁺, Gd³⁺, Nd³⁺, Pr³⁺, Tb³⁺, Pr³⁺, Pm³⁺, Sm³⁺, Eu²⁺, Dy³⁺, Ho³⁺, Er³⁺, Tm³⁺, Yb³⁺.

7. Biodegradable stent according to claim 5, wherein the metal is selected from the group consisting of magnesium, calcium, manganese, iron, zinc, silicon, yttrium, zirconium and gadolinium.

8. Biodegradable stent according to claim 1, wherein the biodegradable coating consists of at least one of the following biodegradable substances or of mixtures of the following biodegradable substances: polydioxanone, polycaprolactone, polygluconate, poly(lactic acid) polyethylene oxide copolymer, modified cellulose, polyhydroxybutyrate, polyamino acids, polyphosphate ester, polyvalerolactone, poly-ε-decalactone, polylactonic acid, polyglycolic acid, polylactides, polyglycolides, copolymers of the polylactides and polyglycolides, poly_ε-caprolactone, polyhydroxybutyric acid, polyhydroxybutyrates, polyhydroxyvalerates, polyhydroxybutyrate-co-valerate, poly(1,4-dioxane-2,3-one), poly(1,3-dioxane-2-one), poly-para-dioxanone, polyanhydrides, polymaleic acid anhydrides, polyhydroxy methacrylates, fibrin, polycyanoacrylate, polycaprolactone dimethylacrylates, poly-β-maleic acid, polycaprolactone butyl acrylates, multiblock polymers from oligocaprolactonediols and oligodioxanonediols, polyether ester multiblock polymers from PEG and poly(butylene terephthalates), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate), polyorthoesters, polyglycolic acid trimethyl carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide, soft polyurethanes, polyurethanes having amino acid residues in the backbone, polyetheresters such as polyethylene oxide, polyalkene oxalates, polyorthoesters as well as copolymers thereof, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivates thereof, heparins, chondroitin sulfate, dextran, β-cyclodextrins, copolymers with PEG and polypropylene glycol, gum arabic, guar, gelatin, collagen N-hydroxysuccinimide, lipids, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylenes, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, EPDM gums, silicones such as polysiloxanes, polydimethylsiloxanes, polyvinyl halogens and copolymers, cellulose ethers, cellulose triacetates, chitosans and copolymers and/or mixtures of the aforementioned polymers.

9. Biodegradable stent according to claim 8, wherein the biodegradable coating consists of a polydioxanone, polycaprolactone, polygluconate, poly(lactic acid) polyethylene oxide copolymer, modified cellulose, collagen, polyhydroxybutyrate, polyanhydride, polyphosphoester, polyester, polyamino acids, polylactides, polyglycolides, poly_ε-caprolactone and/or polyphosphate ester.

10. Biodegradable stent according to claim 1, wherein micropores, holes, openings, channels or other ion-permeable structures are included in the outer polymeric coating.

11. Biodegradable stent according to claim 10, wherein the micropores, holes, openings or channels are filled with an anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent or with a composition containing an anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent.

12. Biodegradable stent according to claim 1, wherein the outer polymeric coating contains at least one anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent.

13. Biodegradable stent according to claim 11, wherein the at least one anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent is selected from the group comprising:
abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, bombrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside la, 4-hydroxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, inhibitor 2□ of smc proliferation, mitoxanthrone, mycophenolate c-myc antisense, b-myc antisense, β-lapachone, podophyllotoxin, podophyllic acid 2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, NFkB, angiopeptine, monoclonal antibodies inhibiting muscle cell proliferation, bFGF antagonists, probucol, prostaglandins, 1-hydroxy-11-methoxycanthin-6-one, scopoletin, NO donors such as pentaerythritol tetranitrate and sydnonimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof such as 6-α-hydroxy-paclitaxel, taxoteres, carbon suboxides (MCS) and macrocylic oligomers thereof, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF 1, active agents from the group of antibiotics such as cefadroxil, cefazolin, cefaclor, cefoxitin, tobramycin, gentamicin, penicillins such as dicloxacillin, oxacillin, sulfonamides, metronidazole, enoxaparin, desulfated and N-reacetylated heparin, tissue plasminogen activator, GpIIb/IIIa platelet membrane receptor, antibodies to factor Xa inhibitor, heparin, hirudin, r-hirudin, PPACK, protamine, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators such as p65, NF-kB or Bcl-xL antisense oligonucleotides, halofuginone, nifedipine, tocopherol, tranilast, molsidomine, tea polyphenols, epicatechin gallate, epigallocatechin gallate, leflunomide, etanercept, sulfasalazine, etoposide, dicloxacylline, tetracycline, triamcinolone, mutamycin, procainimide, retinoic acid, quinidine, disopyrimide, flecamide, propafenone, sotalol, natural and synthetically obtained steroids such as inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, clotrimazole, flucytosine, griseofulvin, ketoconazole, miconazole, nystatin, terbinafine, antiprotozoal agents such as chloroquine, mefloquine, quinine, moreover natural terpenoids such as hippocaesculin, barringtogenol-C21-angelate, 14-dehydroagmstistachin, agroskerin, agrostistachin, 17-hydroxyagrostistachin, ovatodiolids, 4,7-oxycycloanisomelic acid, baccharinoids B1, B2, B3 and B7, tubeimoside, bruceantinoside C, yadanziosides N and P, isodeoxyelephantopin, tomenphantopin A and B, coronarin A, B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, maquiroside A, marchantin A, maytansin, lycoridicin, margetine, pancratistatin, liriodenine, bisparthenolidine, oxoushinsunine, periplocoside A, ursolic acid, deoxypsorospermin, psychorubin, ricin A, sanguinarine, manwu wheat acid, methylsorbifolin, chromones of spathelia, stizophyllin, mansonine, strebloside, dihydrousambaraensine, hydroxyusambarine, strychnopentamine, strychnophylline, usambarine, usambarensine, liriodenine, oxoushinsunine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

14. A biodegradable stent according to claim 1 wherein the biodegradable stent is a stent for blood vessels, the urinary tract, the airways, oesophagus, bile ducts or the intestinal tract.

15. Biodegradable stent according to claim 12, wherein the at least one anti-inflammatory, cytostatic, cytotoxic, antiproliferative, anti-microtubuli, antiangiogenic, antirestenotic (anti-restenosis), antifungicide, antineoplastic, antimigrative, athrombogenic and/or antithrombogenic agent is selected from the group comprising:
abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, bombrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a, 4-hydroxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, inhibitor 2□ of smc proliferation, mitoxanthrone, mycophenolate c-myc antisense, b-myc antisense, β-lapachone, podophyllotoxin, podophyllic acid 2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, NFkB, angiopeptine, monoclonal antibodies inhibiting muscle cell proliferation, bFGF antagonists, probucol, prostaglandins, 1-hydroxy-11-methoxycanthin-6-one, scopoletin, NO donors such as pentaerythritol tetranitrate and sydnonimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof such as 6-α-hydroxy-paclitaxel, taxoteres, carbon suboxides (MCS) and macrocylic oligomers thereof, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF 1, active agents from the group of antibiotics such as cefadroxil, cefazolin, cefaclor, cefoxitin, tobramycin, gentamicin, penicillins such as dicloxacillin, oxacillin, sulfonamides, metronidazole, enoxaparin, desulfated and N-reacetylated heparin, tissue plasminogen activator, GpIIb/IIIa platelet membrane receptor, antibodies to factor Xa inhibitor, heparin, hirudin, r-hirudin, PPACK, protamine, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators such as p65, NF-kB or Bcl-xL antisense oligonucleotides, halofuginone, nifedipine, tocopherol, tranilast, molsidomine, tea polyphenols, epicatechin gallate, epigallocatechin gallate, leflunomide, etanercept, sulfasalazine, etoposide, dicloxacylline, tetracycline, triamcinolone, mutamycin, procainimide, retinoic acid, quinidine, disopyrimide, flecamide, propafenone, sotalol, natural and synthetically obtained steroids such as inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, clotrimazole, flucytosine, griseofulvin, ketoconazole, miconazole, nystatin, terbinafine, antiprotozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceantinoside C, yadanziosides N and P, isodeoxyelephantopin, tomenphantopin A and B, coronarin A, B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, maquiroside A, marchantin A, maytansin, lycoridicin, margetine, pancratistatin, liriodenine, bisparthenolidine, oxoushinsunine, periplocoside A, ursolic acid, deoxypsorospermin, psychorubin, ricin A, sanguinarine, manwu wheat acid, methylsorbifolin, chromones of spathelia, stizophyllin, mansonine, strebloside, dihydrousambaraensine, hydroxyusambarine, strychnopentamine, strychnophylline, usambarine, usambarensine, liriodenine, oxoushinsunine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.
